(19) United States
(12) Patent Application Publication
(10) Pub. No.: US 2017/0188938 A1
(43) Pub. Date: Jul. 6, 2017

(54) SYSTEM AND METHOD FOR MONITORING SLEEP OF A SUBJECT

(71) Applicant: Huckleberry Labs, Inc., Irvine, CA (US)

(72) Inventors: Jessica Toh, Irvine, CA (US); Daniel Huang, Irvine, CA (US); Kuan Hsieh, Cambridge (GB); Seng Oon Toh, Irvine, CA (US)

(21) Appl. No.: 15/399,326

(22) Filed: Jan. 5, 2017

Related U.S. Application Data
(60) Provisional application No. 62/274,931, filed on Jan. 5, 2016.

(51) Int. Cl. A61B 5/00; A61B 5/11; A61B 5/01

(52) U.S. Cl. CPC A61B 5/4809; A61B 5/0064; A61B 5/015; A61B 5/1123; A61B 5/0022; H04W 88/02

(57) ABSTRACT

A method and system for monitoring sleep of a subject is disclosed. In one aspect, the sleep of the subject is monitored using a sensor unit including infrared array sensors for sleep tracking, which is used to track motion and also to detect presence of the subject. Further, the environment surrounding the subject is monitored. Subsequently, sleep data and environment data is generated and stored on a memory of the sensor unit. The present disclosure makes recommendations to the carer to improve the sleep quality of the child based upon the sleep data and environmental data via a portable interaction device such as a mobile phone.

### TECHNICAL FIELD

The present disclosure relates generally to a sleep monitor system; and more specifically, to a sleep monitor system and method for indicating sleep and environment related status of a subject to a user.

### BACKGROUND

Sleep disorders are one of the most commonly occurring disorders among a large section of a society. For example, as per a sleep disorder study, approximately 36% of 3-4 year olds wake up every night requiring assistance, and the percentage of younger children requiring assistance is even higher. These disturbances affect the sleep of other family members also. As a result, a large population suffers from chronically disrupted sleep. Further, long-term consequences of chronically disrupted sleep in children include slow growth, chronic irritability, behavioral problems (e.g., aggressiveness, hyperactivity, and poor impulse control), poor school performance, family disruption, and maternal depression.

Various solutions exist to address the sleep disorders. According to one known solution, sleep consultants are hired to address the sleep related disorders. Generally the sleep consultants are expensive and of varying skill levels. In another known solution, a polysomnography test is conducted to determine cause of the sleep disorders. However, the polysomnography test requires an expensive and inconvenient overnight stay at a clinic. In a yet another known solution, a body worn actigraphy unit is used to monitor human rest/activity cycles that can cause sleep disorders. However, this solution requires that the subject wear the device. The subject, especially a young child, may not like wearing the device or forget to put it on. Further, various under-mattress sensors can be installed to monitor sleeping behavior of the subject.

However, these under-mattress sensors are riddled with false positive outputs because these sensors do not automatically differentiate between scenarios such as when the subject is present but not moving, and when the subject is not present. In addition, these sensors can be subjected to urine, chewing, accidental laundering, or can cause discomfort.

Therefore, there is a need for efficiently monitoring sleeping behavior.

### SUMMARY

The present disclosure seeks to provide a computer-implemented method for recognizing sleep of a subject.

According to one disclosed method for monitoring sleep of a subject, actions include monitoring sleep with a sensor unit having infrared array sensing for sleep tracking which is usable to track motion and also to detect presence; with the sensor unit, converting the information gained through monitoring sleep into sleep data; with the sensor unit, monitoring the environment during sleep; with the sensor unit, converting the information gained through monitoring the environment during sleep to environmental data; combining the sleep data with the environmental data on a memory component of the sensor unit; and making recommendations to the caregiver to improve the sleep quality of the child based upon the sleep data and environmental data via a portable interaction device such as a mobile phone.

According to one disclosed method for recognizing sleep in the a subject, actions include collecting presence and motion data from a sensor unit; transmitting the presence and motion data from the sensor unit to a processing unit; with the processing unit, determining whether the presence and motion data suggest presence of the subject; when the presence and motion data suggest the presence of the subject, determining, with the processing unit, whether a full window of motion data is available; when a full window of motion data is not available, determining, with the processing unit, whether motion single point value is above a threshold; when the motion single point value (mspv) is above the mspv threshold, yielding the conclusion that the subject is asleep; when a full window of motion data is available, computing, with the processing unit, a mean, standard deviation, natural log, a number of events greater than the sum of the mean and a motion data threshold and a maximum motion value over a time window; and processing the mean, standard deviation, natural log, number of events greater than the sum of the mean and motion data threshold and maximum motion value over a time window in accordance with a machine learning model; determining whether the result of the machine learning model processing is a 0 or a 1; and when the result of the machine learning model processing is a 1, yielding the conclusion that the subject is asleep.

Optionally, collecting presence data further comprises collecting infrared images and removing inanimate heat sources from the infrared images. The step of removing inanimate heat sources from the infrared images further comprises filtering the infrared images for each pixel across time using a median filter and a moving average filter; finding an average temperature of the infrared images per frame and filtering the average across time using a median filter; finding time and location of inanimate heat sources in the presence data; masking out inanimate heat sources in the infrared images; and setting the temperature of a masked region to the mean temperature of regions not masked.

Further, the step of collecting presence data comprises performing 2-dimensional 3rd order spline interpolation on the infrared images and determining presence status for the subject.

The step of determining presence status for the subject further comprises determining a temperature gradient image with a sobel operator; determining the number of pixels having temperature above a temperature gradient threshold; and determining whether the number of pixels having temperature above the gradient threshold is greater than a pixel number threshold. Further, presence data is yielded when the number of pixels having temperature above the gradient threshold is greater than the pixel number threshold. Alternatively, no presence data is yielded when the number of pixels having temperature equal to or less than the gradient threshold is not greater than a pixel number threshold.

Optionally, the step of determining presence status for the subject further comprises computing logistic regression parameters; multiplying the logistic regression parameters with corresponding coefficients; processing the products of the logistic regression parameters with the corresponding coefficients with a logistic function; yielding the presence data when the result of the logistic function is greater than or equal to 0.5. and yielding no presence data when the result of the logistic function is less than 0.5.

More optionally, the step of determining the presence status for the subject further comprises performing blob detection; removing background from the infrared images with a dilation technique; finding an Otsu threshold and removing pixels below the Otsu threshold; detecting blobs in the infrared images; calculating sizes of the detected blobs; calculating blob mean temperature; yielding the presence data when at least one of the calculated sizes is greater than a blob size threshold and the difference between the blob mean temperature and a background mean temperature is greater than a temperature difference threshold; and yielding no presence data when none of the calculated sizes is greater than a blob size threshold and the difference between the blob mean temperature and a background mean temperature is not greater than a temperature difference threshold.

Further, the step of collecting presence data further comprises checking and updating presence data.

Optionally, the step of checking and updating presence data further comprises determining whether there has been a change in presence status; calculating past and future motion data when there has been a change in presence status; determining from the past and future motion data whether presence status has changed from no presence to presence; determining whether the future motion exceeds a threshold when the presence status has changed from no presence to presence; detecting any condition of no presence in the following 10 frames when the future motion does not exceed the threshold; undoing any changes in presence when a condition of no presence is detected in the following 10 frames; determining whether the past motion exceeds a threshold when the presence status has not changed from no presence to presence; detecting any condition of presence in the following 10 frames when the past motion does not exceed the threshold; and undoing any changes in presence when a condition of no presence is detected in the following 10 frames.

According to another disclosed method for monitoring sleep of a subject, actions include tracking motion and detecting presence of a subject; converting the tracked motion and presence detections into sleep data; monitoring an environment of the subject during when presence is detected; converting information collected through monitoring the environment into environmental data; combining the sleep data with the environmental data on a memory; and making recommendations to the caregiver to improve the sleep quality of the child based upon the sleep data and environmental data via a portable interaction device such as a mobile phone.

According to another method for recognizing sleep in a subject, actions include collecting presence and motion data from a sensor unit; transmitting the presence and motion data from the sensor unit to a processing unit; with the processing unit, determining whether the presence and motion data suggest presence of the subject; when the presence and motion data do not suggest the presence of the subject, yielding the conclusion that the subject is awake; when the presence and motion data do suggest the presence of the subject, determining, with the processing unit, whether a full window of motion data is available; when a full window of motion data is not available, determining, with the processing unit, whether motion single point value is above a threshold; when the motion single point value is not above a mspv threshold, yielding the conclusion that the subject is awake; and when the motion single point value is above the mspv threshold, yielding the conclusion that the subject is awake; when a full window of motion data is available, computing, with the processing unit, a mean, standard deviation, natural log, a number of events greater than the sum of the mean and a motion data threshold and a maximum motion value over a time window; and processing the mean, standard deviation, natural log, number of events greater than the sum of the mean and motion data threshold and maximum motion value over a time window in accordance with a machine learning model; determining whether the result of the machine learning model processing is a 0 or a 1; when the result of the machine learning model processing is a 0, yielding the conclusion that the subject is awake; and when the result of the machine learning model processing is a 1, yielding the conclusion that the subject is asleep.

### BRIEF DESCRIPTION OF THE FIGURES

The summary above, as well as the following detailed description of illustrative embodiments, is better understood when read in conjunction with the appended drawings. For the purpose of illustrating the present disclosure, example constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 schematically illustrates an example sleep monitor system, in accordance with an embodiment of the present disclosure.

FIG. 2 schematically illustrates an example processing unit of the sleep monitor system, in accordance with an embodiment of the present disclosure.

FIG. 3 illustrates an example computer-implemented method for monitoring sleep of a subject, in accordance with an embodiment of the present disclosure.

FIG. 4 illustrates an example computer-implemented method for recognizing sleep in the subject, in accordance with an embodiment of the present disclosure.

FIG. 5 illustrates an example computer-implemented method for collecting presence and motion data from a sensor unit of the sleep monitor system, in accordance with an embodiment of the present disclosure.

FIG. 6 illustrates an example computer-implemented method for removing inanimate heat sources from infrared images, in accordance with an embodiment of the present disclosure.

FIG. 7 illustrates an example computer-implemented method for detecting presence of the subject, in accordance with an embodiment of the present disclosure.

FIG. 8 illustrates an example computer-implemented method for determining temperature gradient image with a sobel operator, in accordance with an embodiment of the present disclosure.

FIG. 9 illustrates an example computer-implemented method for computing logistic regression parameters, in accordance with an embodiment of the present disclosure.

FIG. 10 illustrates an example computer-implemented method for performing blob detection, in accordance with an embodiment of the present disclosure.

FIG. 11 illustrates an example computer-implemented method for performing presence detection using a convolutional neural network, in accordance with an embodiment of the present disclosure.

FIG. 12 illustrates an example computer-implemented method for checking and updating presence data, in accordance with an embodiment of the present disclosure.

FIG. 13 illustrates an example data flow block diagram corresponding to a computer-implemented method for providing recommendations to a user to improve sleep quality of the subject, in accordance with an embodiment of the present disclosure.

### DETAILED DESCRIPTION

The following detailed description illustrates embodiments of the present disclosure and manners by which they can be implemented. Although the best mode of carrying out the present disclosure has been disclosed, those skilled in the art would recognize that other embodiments for carrying out or practicing the present disclosure are also possible.

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

Embodiments of the present disclosure provide a computer-implemented method for monitoring and recognizing sleep of a subject.

Additionally, embodiments of the present disclosure provide a sleep monitor system for monitoring and recognizing sleep of the subject.

Embodiments of the present disclosure substantially eliminate, or at least partially address, problems in the prior art, and provide a user such as a caregiver access to information regarding sleeping behavior of a subject so that the user can improve the sleep quality of the subject by using the sleep monitor system and methods for monitoring and recognizing the sleep as disclosed herein.

A sleep monitor system of according to the present disclosure is configured to implement a method for enabling the user to monitor sleep of the subject through use of an infrared array sensor or a video camera, which can be adapted to track motion as well as presence of a subject during the sleep. Further, the sleep monitor system is configured to determine data related to an environment surrounding the subject. The sleep monitor system converts the information collected from the infrared array sensor or the video camera into sleep data and environment-related information into environmental data. The sleep monitor system processes the sleep data and the environmental data in order to generate recommendations for the user so that the user can take effective steps to improve the quality of the sleep of the subject. The sleep monitor system can be configured to send the recommendations to the user directly to a communication device of the user.

The sleep monitor system is configured as a contactless device not required to be worn by a subject in contrast to known sleep monitoring devices. Further, the sleep monitor system is not required to be placed on or under a subject's bed. As a result, the sleep monitor system does not cause any discomfort to the subject and is not subjected to bodily fluids or tampering by the subject during night. The sleep monitor system is configured to be operated on a battery and, being portable, allows the user to move the sleep monitor system to different positions in order to conform to changing needs of the subject.

The sleep monitor system can be operated in manual or automatic modes. During the automatic mode of the operation, the sleep monitor system automatically collects data for example, sleep and environment data without requiring any input from the user. As a result, accuracy of the sleep monitor system is significantly improved by complete and detailed information regarding sleeping habits of the subject. Consequently, the sleep monitor system can generate accurate recommendations to the user on identifying sleep related issues of the subject. For example, in order to forecast a subject being overtired (a common contributor to disrupted sleep); a complete and accurate set of sleep data is required. Due to availability of complete data, the sleep monitor system can indicate to the user that the subject is become overtired and the user may be required to provide consultation to the subject in accordance with how overtired the subject is. In automatic mode the sleep monitor system provides a user-friendly device, which can be installed and used by the user without professional efforts.

Further, a sensing unit of the sleep monitor system is configured to store sleep data and environmental data within an internal memory and thus, does not rely on a constant internet connection to send sleep data and environmental data to an external processing unit. The sensing unit is configured to internally process the stored data and recommend to the user steps that may be taken to improve the subject's sleep quality. Additionally, the sensing unit is configured to transmit sleep data and environment data to the external processing unit for storing in a database for later, further analysis. The sensing unit may synchronize the data stored in the memory with the memory of the database. Synchronization can happen automatically whenever the sensing unit discovers an external network providing connectivity to the processing unit.

Further, the processing unit of the sleep monitor system is configured to receive sleep data and environmental data from a plurality of sensing units assigned to the plurality of subjects respectively. As a result, the processing unit becomes a data aggregator for the sleep data of the plurality of subjects and the processing unit is configured to provide trends in the sleeping habits, parameters indicating quality of sleep, and suggest sleeping disorders of a category as may be specified by the user. Further, the processing unit can use the data stored in the database to improve recommendations provided to the user for a particular subject.

The sleep monitor system and method of recommending the user as disclosed herein can be used for other medical and general health services which may benefit from recommendations based on the sleep data and the environmental data. For example, recommendations may be used to proactively respond to medical emergencies such as intervention in circumstances where regular movement during the night is required to be monitored so that any health issues can be immediately communicated to the user, on occurrence of seizures during sleep or while determining effect of medication on sleep of the subject.

Additional aspects, advantages, features and objects of the present disclosure will be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

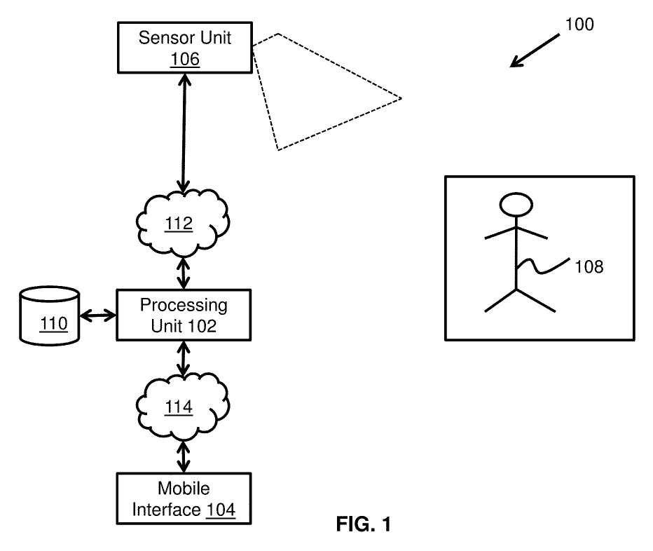

Referring now to the drawings, particularly by their reference numbers, FIG. 1 schematically illustrates an example sleep monitor system 100 in accordance with an embodiment of the present disclosure. The sleep monitor system 100 includes a processing unit 102, a mobile interface 104 and a sensor unit 106 configured to communicatively couple to each other. Sensor unit 106 is configured to monitor sleep data and environmental data pertaining to a subject 108 sleeping on a support for example, a bed.

As illustrated in FIG. 1, the sensor unit 106 is communicatively coupled to the processing unit 102 via a communication network 112. Communication network 112 may be arranged as a collection of individual networks, interconnected with each other and functioning as a single large network. Such individual networks may be wired, wireless, or a combination thereof. Examples of such individual networks include, but are not limited to, Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, and Worldwide Interoperability for Microwave Access (WiMAX) networks.

Sensor unit 106 transmits information related to sleep data and environmental data for the subject 108 so that the processing unit 102 can store the information corresponding to the subject 108 in a database 110. Processing unit 102 can include a web server, an analytics server, and a statistics server, which are configured to process the sleep data and the environmental data to deliver recommendations to the user. In an embodiment, the database 110 and the processing unit 102 may be implemented in various ways, depending on various possible scenarios. In one example scenario, processing unit 102 and the database 110 may be implemented by way of a spatially collocated arrangement of the processing unit 102 and the database 110. In another example scenario, the processing unit 102 and the database 110 may be implemented by way of a spatially distributed arrangement of the processing unit 102 and the database 110 via a communication network 114. In yet another example scenario, the processing unit 102 and the database 110 may be implemented via cloud computing services.

Further, the processing unit 102 is connected to the mobile interface 104 via a communication network 114 which can be a collection of individual networks, interconnected with each other and functioning as a single large network. Such individual networks may be wired, wireless, or a combination thereof. Examples of such individual networks include, but are not limited to, Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, and Worldwide Interoperability for Microwave Access (WiMAX) networks.

Mobile interface 104 can be an interface selected from an interface of at least one of: smart telephones, Mobile Internet Devices (MIDs), tablet computers, Ultra-Mobile Personal Computers (UMPCs), tablet computers, Personal Digital Assistants (PDAs), web pads, Personal Computers (PCs), handheld PCs, laptop computers, desktop computers, Network-Attached Storage (NAS) devices, large-sized touch screens with embedded PCs, and interactive entertainment devices, such as game consoles, Television (TV) sets and Set-Top Boxes (STBs).

In an embodiment, the user may register the mobile interface 104 with the processing unit 102 and the processing unit 102 stores information such as mobile number, a user's identification, an identification of the sensor unit 106 assigned to the user's mobile interface 104 and other related information so as to identify the user. In an embodiment, the user may install application software on the mobile interface 104 so that the application software is configured to directly communicate with the processing unit 102 via the network 114. The application software automatically transfers information associated with the user to the processing unit 102. The processing unit 102, upon analysis of the sleep data and the environmental data associated with the subject 108, transmits recommendations to the mobile interface 104. As a result, the user is able to improve the sleep quality of the subject 108 based on the recommendations.

In an embodiment, the sleep monitor system 100 is configured to provide real time recommendations to the user regarding the sleep quality of the subject 108. During proper network connectivity between the sensor unit 106 and the processing unit 102, the sensor unit 106 is configured to send regular updates of the sleep data and the environmental data to the processing unit 102. The processing unit 102 performs the analysis of the received sleep data and the environmental data on real time basis and accordingly, the user will be able to receive recommendations in real time. Otherwise, the sleep monitor system 100 is configured to operate in an offline or intermittent access mode wherein the sensor unit 106 collects and stores the sleep data and the environmental data in its internal memory and, upon finding a proper connectivity with the processing unit 102; the sensor unit 106 transmits the data to the processing unit 102. As a result, the sleep monitor system 100 can provide information to the user on a real time basis while a proper connectivity between the sensor unit 106 and the processing unit 102 is available and in a retroactive offline mode too when no or only intermittent connectivity between the sensor unit 106 and the processing unit 102 is available.

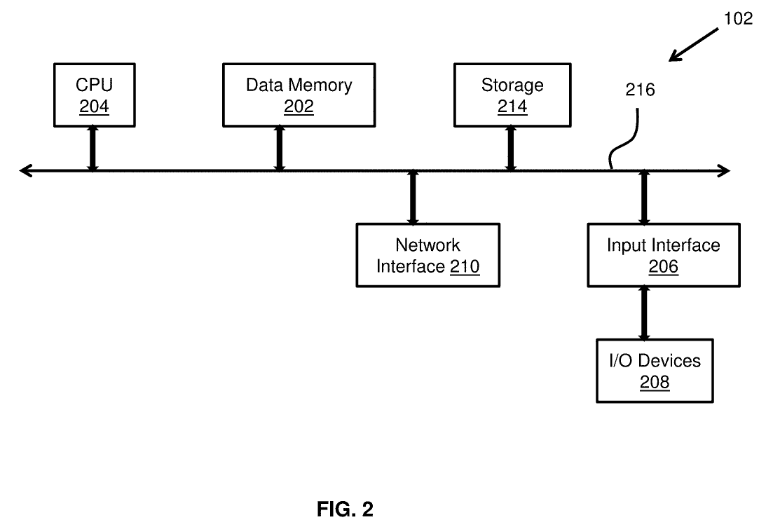

FIG. 2 is a schematic illustration of various components of the processing unit 102, in accordance with an embodiment of the present disclosure. The processing unit 102 includes, but is not limited to, a data memory 202, a computing hardware such as a central processing unit (CPU) 204, an input interface to connect one or more Input/output (I/O) devices 208, a network interface 210, a storage 214, and a system bus 216 that operatively couples various components including the data memory 202, the CPU 204, the I/O devices 208, the network interface 210, the sensors 212 and the storage 214.

The I/O devices 208 include may a display screen for presenting graphical images to a user of the processing unit 102. In some examples, the display screen may be a touch-sensitive display screen that is operable to receive tactile inputs from the user. These tactile inputs may, for example, include clicking, tapping, pointing, moving, pressing and/or swiping with a finger or a touch-sensitive object like a pen. Additionally or alternatively, the I/O devices 208 include a mouse or a joystick that is operable to receive inputs corresponding to clicking, pointing, and/or moving a pointer object on the graphical user interface. The I/O devices 208 may also include a keyboard that is operable to receive inputs corresponding to pushing certain buttons on the keyboard. Additionally, the I/O devices 208 may also include a microphone for receiving an audio input from the user, and a speaker for providing an audio output to the user.

The processing unit 102 also includes a power source for supplying electrical power to the various components of the processing unit 102. The power source may, for example, include a rechargeable battery.

The data memory 202 optionally includes non-removable memory, removable memory, or a combination thereof. The non-removable memory, for example, includes Random-Access Memory (RAM), Read-Only Memory (ROM), flash memory, or a hard drive. The removable memory, for example, includes flash memory cards, memory sticks, or smart cards.

The data memory 202 is configured to store various modules such as a sleep monitor module configured to monitor the sleep of the subject in accordance with the methods as disclosed herein. The sleep monitor module may, for example, be parts of a software product associated with the sleep monitoring and recommendation related features provided by the processing unit 102. Executing the software product on the CPU 204 results in generating recommendations to the user so that the user can improve the sleep quality of the subject.

Moreover, the storage 214 is non-transient data storage medium. The software product, when executed on the CPU 204, is optionally coupled to the storage 214, and is configured to substantially continuously record and update sleep data and environment data for a plurality of subjects in the storage 214.

Furthermore, the network interface 210 optionally allows the processing unit 102 to communicate with other communication devices such as a mobile device of the user so that the processing unit 102 can recommend the actions that the user may be required to take in order to improve sleep quality of the subject. Additionally, the network interface 210 may allow the processing unit 102 to access an external database in order to aggregate sleep data and environmental data received from the plurality of sensor units of the sleep monitor system 100.

The processing unit 102 is optionally implemented by way of at least one of: a mobile phone, a smart telephone, an MID, a tablet computer, a UMPC, a tablet computer, a PDA, a web pad, a PC, a handheld PC, a laptop computer, a desktop computer, an NAS device, a large-sized touch screen with an embedded PC, an analytics server, a web server and the like.

When executed on the CPU 204, the sleep monitoring module allows the processing unit 102 to receive presence and motion data of the subject from the sensor unit 106. The processing unit 102 is configured to determine the presence of the subject 108 based on the received presence and the motion data. The processing unit 102 may indicate to the user that the subject 108 is awake if an analysis of the presence and motion data does not suggest the presence of the subject 108.

The sleep monitoring module is configured to allow the processing unit 102 to determine an availability of a full window of motion data when the presence and motion data suggest the presence of the subject 108. The processing unit 102 is further configured to determine whether a motion single point value is above a threshold when the full window of motion data is not available. Subsequently, the processing unit 102 yields a conclusion that the subject 108 is awake when the motion single point value is not above an mspv threshold.

However, the processing unit 102 is configured to compute a mean, standard deviation, natural log, a number of events greater than the sum of the mean and a motion data threshold and a maximum motion value over a time window when the full window of motion data is available. Subsequently, the processing unit 102 is configured to process the mean, standard deviation, natural log, number of events greater than the sum of the mean and motion data threshold and maximum motion value over a time window in accordance with a machine learning model.

Further, the processing unit 102 is configured to determine result of the machine learning model. The processing unit 102 is configured to yield a conclusion that the subject 108 is awake when the result of the machine learning model processing is a 0, and a conclusion that the subject 108 is asleep when the result of the machine learning model processing is a 1. Further, the sleep monitoring module of the processing unit 102 may enable the processing unit 102 to transmit these results to the user on a real time basis.

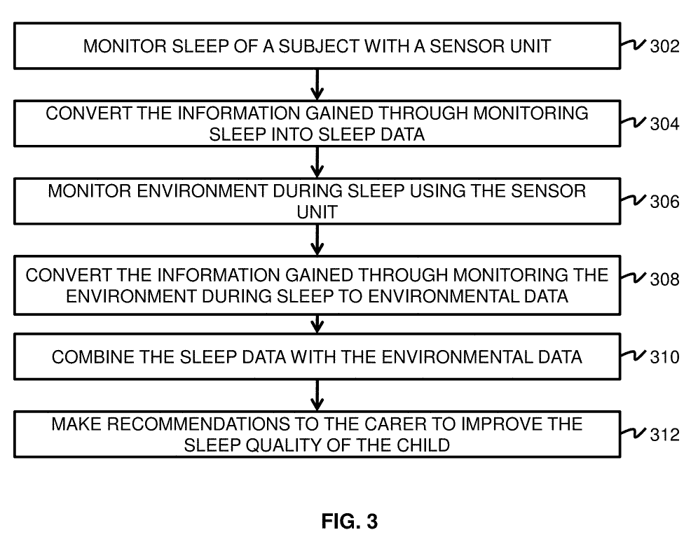

FIG. 3 illustrates an example computer-implemented method 300 for monitoring sleep of a subject, in accordance with an embodiment of the present disclosure. The method is depicted as a collection of actions in a logical flow diagram, which may be implemented in hardware, software, or a combination thereof.

At 302, the method 300 is configured to monitor sleep of a subject with a sensor unit (for example, the sensor unit 106 of FIG. 1). The sensor unit includes an infrared array sensor or a video camera for sleep tracking by tracking motion and presence of the subject 108. Subsequently, at 304, information gained through monitoring sleep is converted into sleep data using the sensor unit.

At 306, the method 300 is configured to monitor environment during sleep using the sensor unit and subsequently, at 308, convert information gained through monitoring the environment during sleep into environmental data using the sensor unit.

At 310, sleep data is combined with the environmental data on a memory component of the sensor unit. At 312, recommendations are made to the caregiver to improve the sleep quality of the subject based upon the sleep data and environmental data via a user mobile device.

The method 300 may include buffering the sleep and environmental data on a memory component of the sensor unit. Subsequently, the method 300 may include synchronizing the buffered sleep and environmental data between the memory component and the processing unit over a network connection. In an embodiment, the synchronization of the data between the memory component and the processing unit may happen through a mobile interface over a network connection. In addition, the method 300 may include aggregating the buffered sleep and environmental data from the processing unit for a plurality of subjects.

The actions 302 to 312 are only illustrative and other alternatives can also be provided where one or more actions are added, one or more actions are removed, or one or more actions are provided in a different sequence without departing from the scope of the claims herein.

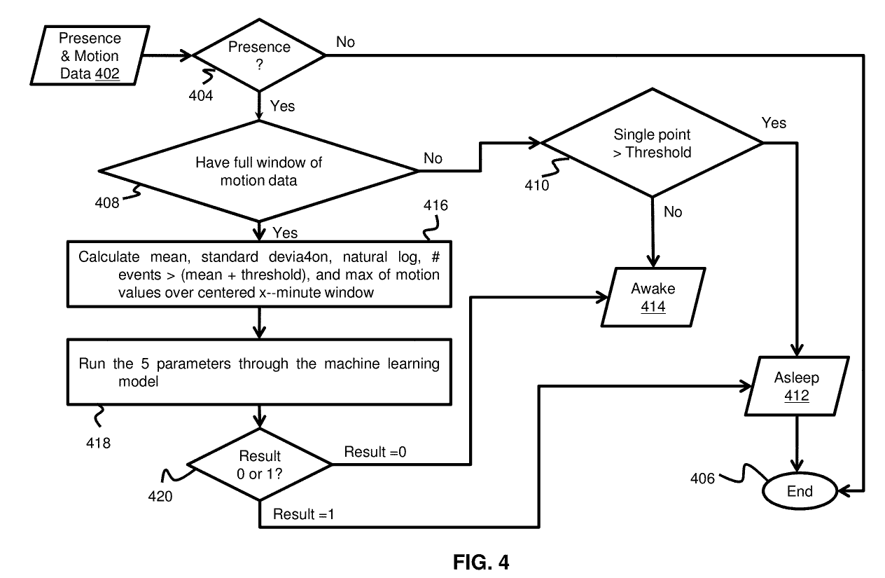

FIG. 4 illustrates an example computer-implemented method 400 for recognizing sleep in the subject, in accordance with an embodiment of the present disclosure. The method is depicted as a collection of actions in a logical flow diagram, which may be implemented in hardware, software, or a combination thereof.

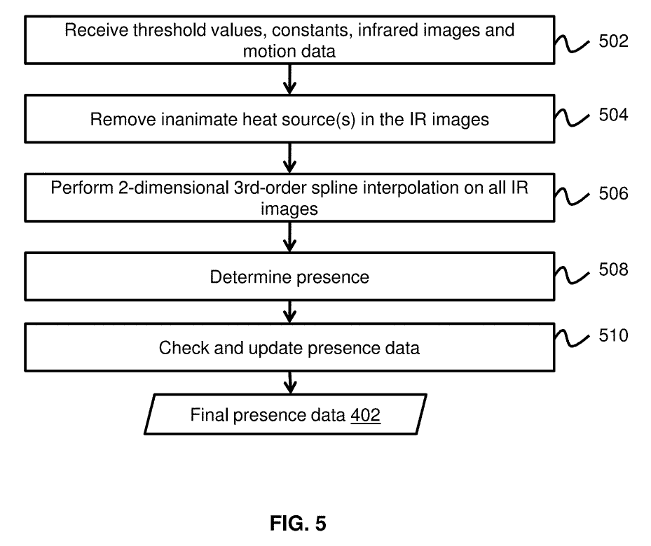

At 402, presence and motion data are collected from a sensor unit and transmitted to a processing unit. A process of collection of the presence and motion data is described by way of example below with reference to FIG. 5.

At 404, the processing unit determines whether the presence and motion data suggest presence of the subject. If it is determined that the subject is not present, the processing unit will continue to make this assessment until presence is detected.

If the processing unit establishes the presence of the subject, at 408, processing unit determines whether a full window of motion data is available. If the full window of the motion data is not available, a determination is made at 410 as to whether a motion single point value is above a threshold value. An output 412 indicating that the subject is asleep is delivered when it is determined that the motion single point value is above the threshold value. An output 414 indicating that the subject is awake is delivered when it is determined that the motion single point value is not above the threshold value.

If the full window of the motion data is available, the processing unit computes, a mean, standard deviation, natural log, a number of events greater than the sum of the mean and a motion data threshold and a maximum motion value over a time window.

At 418, processing unit processes the mean, standard deviation, and natural log, number of events greater than the sum of the mean and motion data threshold and maximum motion value over a time window in accordance with a machine learning model.

At 420, a determination is made as to whether the yield of the machine learning model is a 0 or a 1. If the result is 1, the output 412 indicating that the subject is asleep is delivered. If the result is 0, the output 414 indicating that the subject is awake is delivered.

The actions 404 to 420 are only illustrative and other alternatives can also be provided where one or more actions are added, one or more actions are removed, or one or more actions are provided in a different sequence without departing from the scope of the claims herein.

FIG. 5 illustrates an example computer-implemented method 500 for collecting presence and motion data 402 from the sensor unit, in accordance with an embodiment of the present disclosure. The method is depicted as a collection of actions in a logical flow diagram, which may be implemented in hardware, software, or a combination thereof.

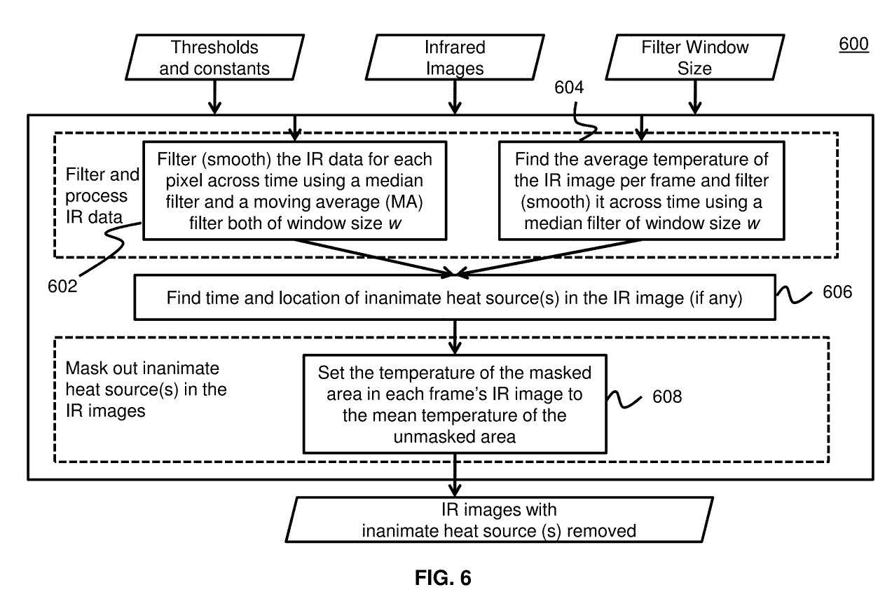

At 502, threshold values, constants, infrared images and motion data corresponding to the subject and for a predetermined amount of time are received. Motion data may include but is not limited to PIR data, video data, use of image data, and sound data. As an example and not as a limitation, the predetermined amount of time can be based on minutes or hours. At 504, inanimate heat sources are removed from the infrared images. A process of removal of inanimate heat sources from the infrared images is described by way of example below with reference to FIG. 6.

At 506, a 2-dimensional 3rd order spline interpolation is performed on the infrared images. Based on the data available from 508, presence status of the subject is determined. A process of determining presence status for the subject is described by way of example below with reference to FIG. 6. At 510, presence and motion data are checked and updated in order to generate final presence and motion data 402 useable in one or more actions of method 400. In an example, final presence and motion data 402 may be employed at 404 of method 400.

The actions 502 to 510 are only illustrative and other alternatives can also be provided where one or more actions are added, one or more actions are removed, or one or more actions are provided in a different sequence without departing from the scope of the claims herein.

FIG. 6 illustrates an example computer-implemented method 600 for removing inanimate heat sources from the infrared images, in accordance with an embodiment of the present disclosure. The method 600 is depicted as a collection of actions in a logical flow diagram, which may be implemented in hardware, software, or a combination thereof.

At 602, the infrared images are filtered for each pixel across time using a median filter and a moving average filter. At 604, an average temperature of the infrared images is found per frame and the average across time is filtered using a median filter. In an embodiment, the median filter and the moving average filter include a filter window size. In an example, the filter window size is the window size of the median filter and the size of the window corresponds to an odd number. In another example, the filter window size is also the window size of the moving average filter, the kernel of which is a uniform distribution of length w and of height 1/w. The w is set to the odd number that gives smoothing effect on the IR data.

At 606, time and location of inanimate heat sources are found in the presence data. At 608, inanimate heat sources are masked out in the infrared images in order to generate infrared images with removed inanimate heat resources. In an embodiment, the temperature of a masked region is set to the mean temperature of regions not masked. As described earlier in the description, the output of the method 600 is useable in one or more actions of method 500, for example at 506.

The actions 602 to 608 are only illustrative and other alternatives can also be provided where one or more actions are added, one or more actions are removed, or one or more actions are provided in a different sequence without departing from the scope of the claims herein.

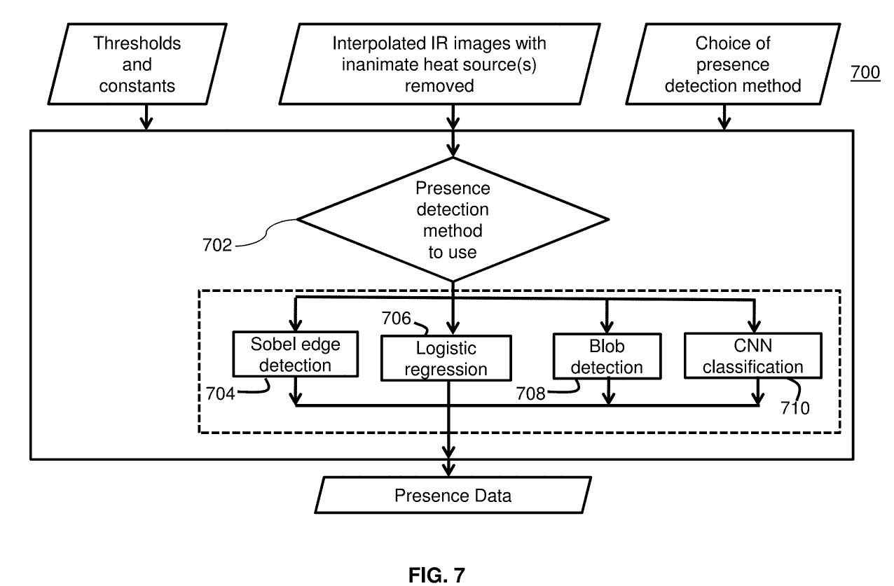

FIG. 7 illustrates an example computer-implemented method 700 for detecting presence of the subject using the output at 506 of the method 500, in accordance with an embodiment of the present disclosure. The method 700 is depicted as a collection of actions in a logical flow diagram, which may be implemented in hardware, software, or a combination thereof.

At 702, a determination is made as to selection of a process for detecting presence of the subject. In an embodiment, any one process out of the processes as indicated at 704, 706, 708 and 710 may be selected.

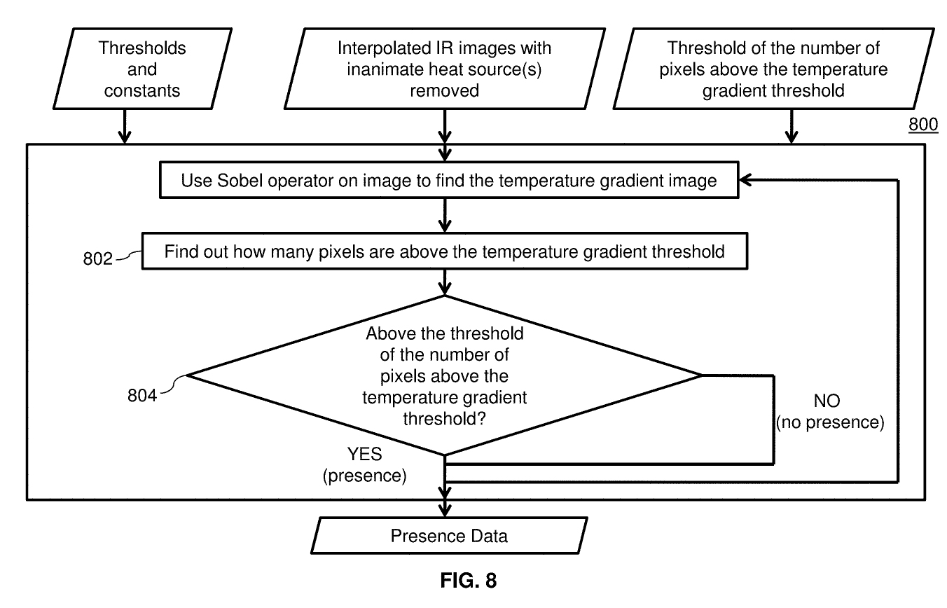

At 704, a temperature gradient image is determined with a sobel operator in order to generate presence data. In an embodiment, the temperature gradient image is determined with sobel operator described by way of example below with reference to FIG. 8. The Sobel operator, sometimes called the Sobel-Feldman operator or Sobel filter, is used in image processing and computer vision, particularly within edge detection algorithms where it creates an image emphasising edges. It is named after Irwin Sobel and Gary Feldman, colleagues at the Stanford Artificial Intelligence Laboratory (SAIL). It was co-developed with Gary Feldman at SAIL. Sobel and Feldman presented the idea of an “Isotropic 3×3 Image Gradient Operator” at a talk at SAIL in 1968. Technically, it is a discrete differentiation operator, computing an approximation of the gradient of the image intensity function. At each point in the image, the result of the Sobel—Feldman operator is either the corresponding gradient vector or the norm of this vector. The Sobel—Feldman operator is based on convolving the image with a small, separable, and integer-valued filter in the horizontal and vertical directions and is therefore relatively inexpensive in terms of computations. On the other hand, the gradient approximation that it produces is relatively crude, in particular for high-frequency variations in the image.

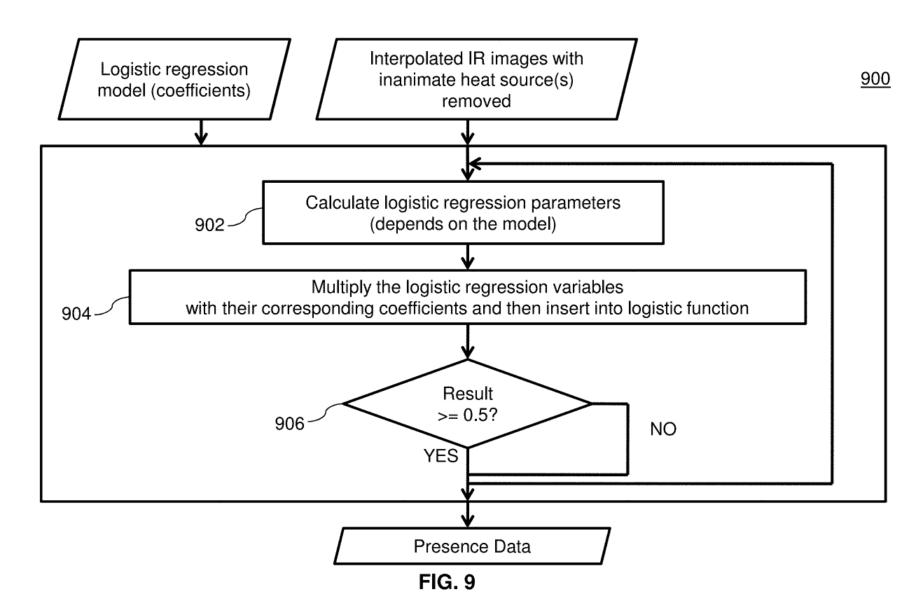

At 706, logistic regression parameters are computed. In an embodiment, logistic regression is computed as described by way of example below with reference to FIG. 9.

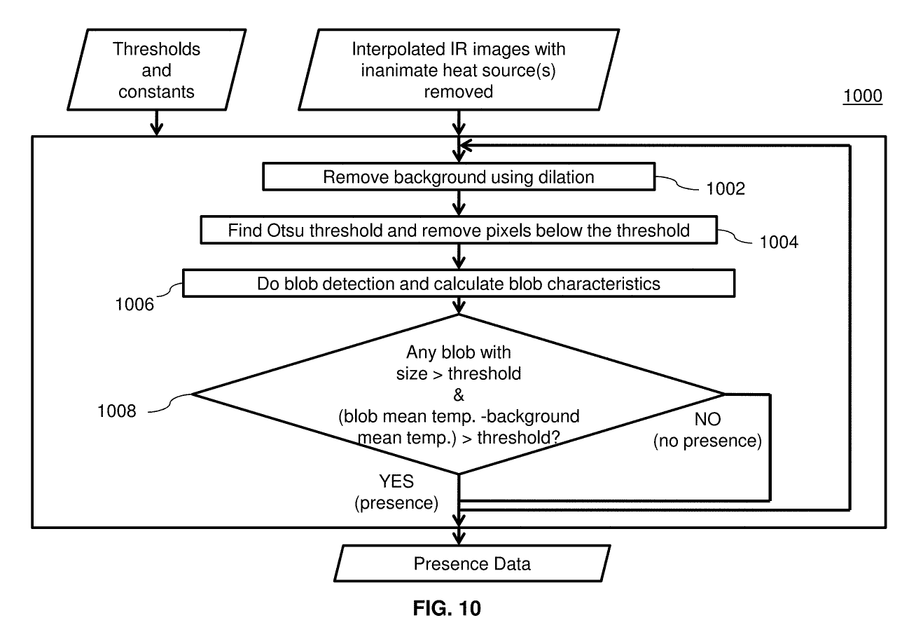

At 708, blob detection is performed. In an embodiment, blob detection may be performed as described by way of example below with reference to FIG. 10. In computer vision, blob detection methods are aimed at detecting regions in a digital image that differ in properties, such as brightness or color, compared to surrounding regions. Informally, a blob is a region of an image in which some properties are constant or approximately constant; all the points in a blob can be considered in some sense to be similar to each other.

Given some property of interest expressed as a function of position on the image, there are two main classes of blob detectors: (i) differential methods, which are based on derivatives of the function with respect to position, and (ii) methods based on local extrema, which are based on finding the local maxima and minima of the function. With the more recent terminology used in the field, these detectors can also be referred to as interest point operators, or alternatively interest region operators.

Blob detection provides complementary information about regions, which is not obtained from edge detectors or corner detectors. Moreover, blob detection as been used to obtain regions of interest for further processing. These regions potentially signal the presence of objects or parts of objects in the image domain with application to object recognition and/or object tracking. In other domains, such as histogram analysis, blob descriptors is also optionally used for peak detection with application to segmentation. Blob descriptors may be used as as main primitives for texture analysis and texture recognition.

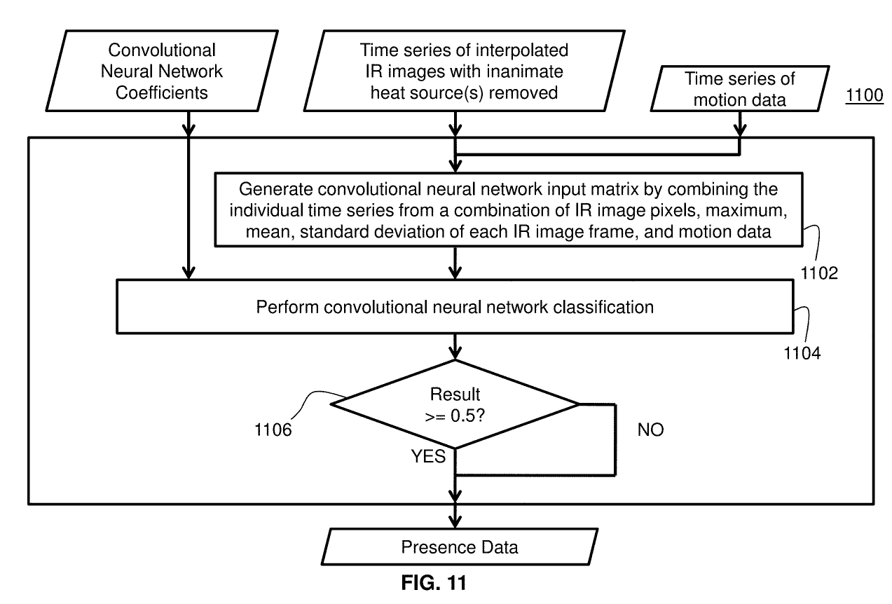

At 710, a convolutional neural network (CNN) classification is performed. In an embodiment, the CNN classification may be performed as described by way of example below with reference to FIG. 11.

The actions 702 to 710 are only illustrative and other alternatives can also be provided where one or more actions are added, one or more actions are removed, or one or more actions are provided in a different sequence without departing from the scope of the claims herein.

FIG. 8 illustrates an example computer-implemented method 800 for determining the temperature gradient image with the sobel operator, in accordance with an embodiment of the present disclosure. The method 800 is depicted as a collection of actions in a logical flow diagram, which may be implemented in hardware, software, or a combination thereof.

At 802, determine the number of pixels having temperature above a temperature gradient threshold is determined. Since a subject's heat blob in the infrared image may have large temperature gradients at the edges, and other places may not have, a threshold can be set that determines whether there is a distinct heat blob or not, which in turn determines the presence of the subject. This threshold is referred to as the temperature gradient threshold. However, the infrared image may have large temperature gradients due to noise and these temperature ingredients may deliver incorrect presence estimates. Therefore, another threshold is defined in order to separate actual heat blobs being detected from spurious noise.

At 804, a determination is made as to whether the number of pixels having temperature above the gradient threshold is greater than a pixel number threshold. Since very few noise pixels may have a large temperature gradient, this reduces the probability of detecting presence detected due to noise.

An output reflecting presence data is delivered when the number of pixels having temperature above the gradient threshold is greater than the pixel number threshold. Otherwise, when the number of pixels having temperature equal to or less than the gradient threshold is not greater than a pixel number threshold no presence data is output.

The actions 802 and 804 are only illustrative and other alternatives can also be provided where one or more actions are added, one or more actions are removed, or one or more actions are provided in a different sequence without departing from the scope of the claims herein.

FIG. 9 illustrates an example computer-implemented method 900 for computing the logistic regression parameters, in accordance with an embodiment of the present disclosure. The method 900 is depicted as a collection of actions in a logical flow diagram, which may be implemented in hardware, software, or a combination thereof.

At 902, logistic regression parameters are calculated. At, the logistic regression parameters are multiplied with corresponding coefficients and the products of the logistic regression parameters are processed with the corresponding coefficients of a logistic function.

At 906, a determination is made as to whether a result at 904 is greater than or equal to a value 0.5. When the result of the logistic function is greater than or equal to 0.5 presence data is output. Otherwise, when the result of the logistic function is less than 0.5. no presence data is output.

The actions 902 to 906 are only illustrative and other alternatives can also be provided where one or more actions are added, one or more actions are removed, or one or more actions are provided in a different sequence without departing from the scope of the claims herein.

FIG. 10 illustrates an example computer-implemented method 1000 for performing the blob detection, in accordance with an embodiment of the present disclosure. The method 1000 is depicted as a collection of actions in a logical flow diagram, which may be implemented in hardware, software, or a combination thereof.

At 1002, background is removed from the infrared images with a dilation technique. At 1004, an Otsu threshold is found and pixels below the Otsu threshold are removed.

At 1006, blobs are detected in the infrared images and sizes of the detected blobs are calculated. At 1008, a determination is made as to whether a blob exists whose size is greater than a threshold blob size. In an embodiment, this threshold is used to determine which blobs captured at 1002 are random noise so that these blobs can be ignored.

In an embodiment, each blob has its own properties such as a size, a maximum temperature, an average temperature and the like. In order for a heat blob to be considered that of the subject, the heat blob must have a relatively large blob area threshold and a high temperature. In other words, the blob is required to have at least a threshold temperature above the background temperature, which is generally computed as a difference between the blob mean temperature and the background mean temperature. Further, the background mean temperature is calculated using the part of the image that is under the Otsu threshold. At 1008, a further determination is made as to whether the difference between blob mean temperature and a background mean temperature is greater than a temperature difference threshold.

When at least one of the calculated sizes is greater than a blob size threshold and the difference between the blob mean temperature and a background mean temperature is greater than a temperature difference threshold, the presence data is output. Otherwise, when none of the calculated sizes is greater than a blob size threshold and the difference between the blob mean temperature and a background mean temperature is not greater than a temperature difference threshold no presence data is output.

The actions 1002 to 1008 are only illustrative and other alternatives can also be provided where one or more actions are added, one or more actions are removed, or one or more actions are provided in a different sequence without departing from the scope of the claims herein.

FIG. 11 illustrates an example computer-implemented method 1100 for performing presence detection using a convolutional neural network that is designed to process time series data. The method 1100 is depicted as a collection of actions in a logical flow diagram, which may be implemented in hardware, software, or a combination thereof.

At 1102, time series data from various input sources are combined together to form an input matrix for the convolutional neural network. In addition, the time series data for the input matrix are generated from each IR image frame by taking the maximum, mean, and standard deviation of the pixels corresponding to each frame. At 1104, convolutional neural network classification is performed on the input matrix using the convolutional neural network coefficients that are used to configure the classifier to detect presence from the time series input matrix.

At 1106, a determination is made as to whether a result of the classifier is greater than or equal to a value 0.5. When the result of the classifier is greater than or equal to 0.5 presence data is output. Otherwise, when the result of the classifier is less than 0.5. no presence data is output.

The actions 1102 to 1106 are only illustrative and other alternatives can also be provided where one or more actions are added, one or more actions are removed, or one or more actions are provided in a different sequence without departing from the scope of the claims herein.

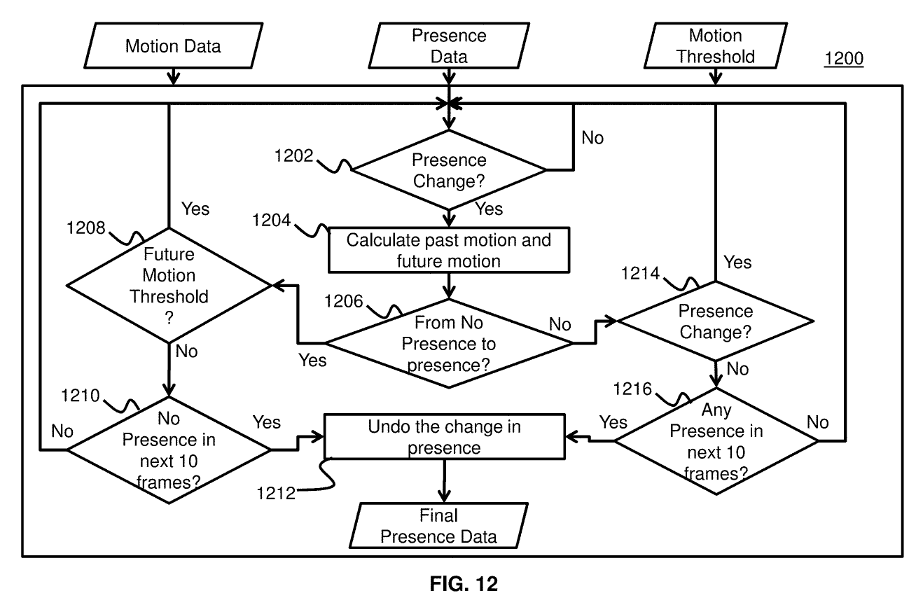

FIG. 12 illustrates an example computer-implemented method 1200 for checking and updating presence data, in accordance with an embodiment of the present disclosure. The method 1200 is depicted as a collection of actions in a logical flow diagram, which may be implemented in hardware, software, or a combination thereof.

At 1202, it is determined whether there has been a change in presence status. When there has been a change in presence status, past and future motion data are calculated at 1204.

At 1206, a determination is made from the past and future motion data as to whether presence status has changed from no presence to presence. When the presence status has changed from no presence to presence, it is determined whether the future motion exceeds a threshold at 1208.

When it is determined that the future motion does not exceed the threshold, any condition of no presence in the following 10 frames is detected at 1210. When a condition of no presence is detected in the following 10 frames, any changes in presence are undone. Otherwise, the determination as to whether there has been a change in presence status is performed again at 1202.

Further, when the presence status has not changed from no presence to presence at 1206, it is determined whether the past motion exceeds a threshold at 1214.

When the post motion does not cross a threshold value, any condition of presence is detected in the following 10 frames at 1216, when the past motion does not exceed the threshold. Any changes in presence are undone at 1212 when a condition of no presence is detected in the following 10 frames. Consequently, any updated presence data is produced.

The actions 1202 to 1216 are only illustrative and other alternatives can also be provided where one or more actions are added, one or more actions are removed, or one or more actions are provided in a different sequence without departing from the scope of the claims herein.

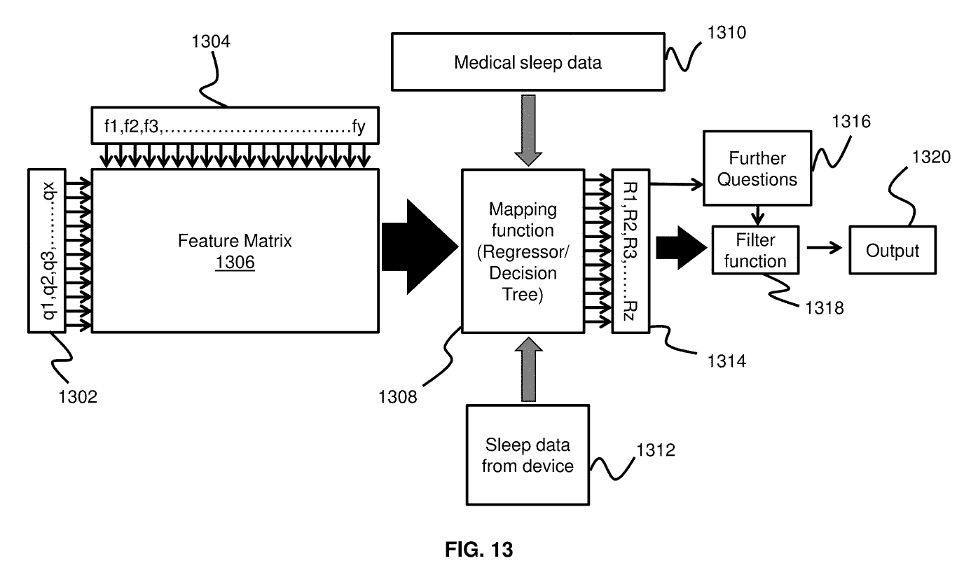

FIG. 13 illustrates an example data flow block diagram 1300 corresponding to a computer-implemented method for providing recommendations to a user to improve sleep quality of the subject, in accordance with an embodiment of the present disclosure. The sleep data is either provided by the sleep monitor system and/or logged by one or more users. The sleep data is analyzed and reported as sleep features functions 1304, examples of which include but are not limited to average time of waking, number of night awakenings each night, and average time gap between naps. When one or more questions are asked to ascertain personal subject traits a subject array 1302 is formed. Examples of obtained subject array 1302 traits include but are not limited to how a child falls asleep at night, reasons for waking up at night, usual dinner time, and parenting style. The sleep feature functions 1304 along with subject array 1302 together form a feature matrix 1306.

In an alternative embodiment, the sleep monitor system is configured to receive questions in a subject array 1302 from various users or from multiple other sleep monitor systems. The questions subject arrays 1302 indicate to concerns raised by the various users in the past to improve sleep quality of their respective subjects. The questions subject arrays 1302 are classified into respective categories using one or more feature functions 1304. As a result, a feature matrix 1306 is created. The feature matrix 1306 indicates a map between the questions subject arrays 1302 and the respective feature functions 1304.

Further, a mapping function 1308 is processed using the feature matrix 1306, medical sleep data 1310, and sleep data 1312 of the subject to generate one or more recommendations 1314 for the user to improve sleep quality of the subject. The user may be asked additional set of questions 1316 in order to identify and address one or more sleep disorders associated with the subject. Based on the responses of the user to the additional set of questions 1316, a filter function 1318 is executed to generate an output 1320. The output 1320 includes recommendations specific to the sleep disorders associated with the subject of the user.

Modifications to embodiments of the present disclosure described in the foregoing are possible without departing from the scope of the present disclosure as defined by the accompanying claims. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A method for monitoring sleep, comprising:
monitoring sleep with a sensor unit including infrared array sensing for sleep tracking which is used to track motion and also to detect presence;
with the sensor unit, converting the information gained through monitoring sleep into sleep data;
with the sensor unit, monitoring the environment during sleep;
with the sensor unit, converting the information gained through monitoring the environment during sleep to environmental data;
combining the sleep data with the environmental data on a memory component of the sensor unit; and
making recommendations to the carer to improve the sleep quality of the child based upon the sleep data and environmental data via a portable interaction device such as a mobile phone.

2. The method as set forth in claim 1, further comprising buffering the sleep and environmental data on the memory component.

3. The method as set forth in claim 2, further comprising synchronizing the buffered sleep and environmental data between the memory component and the processing unit over a network connection.

4. The method as set forth in claim 3, wherein synchronizing further comprises synchronizing between the memory component and the processing unit through a mobile interface over a network connection

5. The method as set forth in claim 3, further comprising aggregating the buffered sleep and environmental data from the processing unit for a plurality of subjects.

6. A method for recognizing sleep in a subject, comprising:
collecting presence and motion data from a sensor unit;
transmitting the presence and motion data from the sensor unit to a processing unit;
with the processing unit, determining whether the presence and motion data suggest presence of the subject;
when the presence and motion data suggest the presence of the subject, determining, with the processing unit, whether a full window of motion data is available;
when a full window of motion data is not available, determining, with the processing unit, whether motion single point value is above a threshold;
when the motion single point value (mspv) is above the mspv threshold, yielding the conclusion that the subject is asleep;
when a full window of motion data is available, computing, with the processing unit, a mean, standard deviation, natural log, a number of events greater than the sum of the mean and a motion data threshold and a maximum motion value over a time window; and
processing the mean, standard deviation, natural log, number of events greater than the sum of the mean and motion data threshold, and maximum motion value over a time window in accordance with a machine learning model;
determining whether the result of the machine learning model processing is a 0 or a 1; and
when the result of the machine learning model processing is a 1, yielding the conclusion that the subject is asleep.

7. The method as set forth in claim 6, wherein collecting presence data further comprises collecting infrared images.

8. The method as set forth in claim 7, collecting presence data further comprises removing inanimate heat sources from the infrared images.

9. The method as set forth in claim 8, wherein removing inanimate heat sources from the infrared images further comprises filtering the infrared images for each pixel across time using a median filter and a moving average filter.

10. The method as set forth in claim 6, wherein collecting presence data further comprises collecting video images.

11. The method as set forth in claim 6, wherein collecting presence data further comprises determining a presence status for the subject, wherein determining the presence status for the subject comprises at least one of:
determining a temperature gradient image by using a sobel operator;
computing logistic regression parameters;
performing blob detection; and
performing a convolutional neural network (CNN) classification.

12. A computer program product comprising a non-transitory computer-readable storage medium storing computer-executable code for recognizing sleep, wherein the code, when executed, is configured to perform the steps of claim 6 with a processor.

13. A method for monitoring sleep, comprising:
tracking motion and detecting presence of a subject;
converting the tracked motion and presence detections into sleep data;
monitoring an environment of the subject during when presence is detected;
converting information collected through monitoring the environment into environmental data;
combining the sleep data with the environmental data on a memory; and
making recommendations to the carer to improve the sleep quality of the child based upon the sleep data and environmental data via a portable interaction device such as a mobile phone.

14. The method as set forth in claim 13, further comprising buffering the sleep and environmental data on the memory.

15. The method as set forth in claim 14, further comprising synchronizing the buffered sleep and environmental data between the memory and a remote processing unit over a network connection.

16. The method as set forth in claim 15, wherein synchronizing further comprises synchronizing the buffered sleep and environmental data between the memory and a mobile interface over a network connection

17. The method as set forth in claim 16, further comprising aggregating the buffered sleep and environmental data from the processing unit for a plurality of subjects.

18. A method for providing recommendations to a user to improve sleep quality of a subject, the method comprising:
storing a plurality of questions received from a plurality of users regarding recommendations to improve sleep quality for a plurality of subjects respectively;
classifying the plurality of questions into respective plurality of categories in order to create at least one matrix indicating a map between at least one question of the plurality of questions and at least one feature; and
processing medical sleep data, sleep data of the subject and the at least one matrix to generate recommendations for the user to improve sleep quality of the subject.

19. The method as set forth in claim 18, further comprising delivering at least one set of questions to the user to address at least one sleep disorder associated with the subject.

20. The method as set forth in claim 18, further comprising executing a filter function to derive at least one recommendation specific to the at least one sleep disorder.
